(19) United States
(12) Patent Application Publication
(10) Pub. No.: US 2016/0338618 A1
(43) Pub. Date: Nov. 24, 2016

(54) METHODS AND SYSTEMS FOR DETERMINING A METABOLIC FUEL TYPE BEING METABOLIZED

(71) Applicant: University of Alaska Fairbanks, Fairbanks, AK (US)

(72) Inventor: Michael B. Harris, Fairbanks, AK (US)

(21) Appl. No.: 14/718,881

(22) Filed: May 21, 2015

(51) Int. Cl. A61B 5/083; A61B 5/00

(52) U.S. Cl. CPC A61B 5/0833; A61B 5/0836; A61B 5/4872

(57) ABSTRACT

Methods and systems for determining a proportion of a metabolic fuel being metabolized are disclosed. A computer can receive concentration measurements of substances exhaled by a user during a time period. The computer can determine a time of interest that occurs during the time period when a change occurs in the proportion of a first metabolic fuel type being metabolized by the user. The change in the proportion can be determined based on the concentration measurements of the substances, such as a respiratory quotient which corresponds to the levels of the first metabolic fuel type and a second metabolic fuel type being metabolized. These proportions can be indicated to the user as well as the time of interest when the change in the proportion occurs in the first metabolic fuel type being metabolized.

### BACKGROUND

Exercise is performed for a number of reasons, including recreation, strength training, muscle conditioning, and cardiovascular fitness. Many exercise to achieve or maintain a body condition such as reducing or maintaining a body fat percentage. With exercise, metabolic processes access a combination of glucose and stored fat for use as a metabolic fuel; consumption of these fuels is initiated during exercise and maintained for a period following exercise. What is lacking is a way to confirm when an exercise, intended to initiate fat metabolism, is sufficient to indicate when a user's metabolism has switched from primarily glucose metabolism to primarily fat metabolism during and following exercise and to track how much fat has been metabolized. These and other shortcomings are addressed in the present disclosure.

### SUMMARY

It is to be understood that both the following general description and the following detailed description are exemplary and explanatory only and are not restrictive. Provided are methods and systems for determining a metabolic fuel type being metabolized by a user.

In an aspect, example methods and systems can comprise receiving concentration measurements of substances exhaled by a user during a time period. The methods and systems can determine a time of interest that occurs during the time period when a change occurs in a proportion of a metabolic fuel type being metabolized by the user. The change in the proportion can be determined based on concentration measurements of substances, such as a respiratory quotient which corresponds to levels of a first metabolic fuel type and a second metabolic fuel type being metabolized. These measurements can be indicated to the user as well as the time of interest when the change in the proportion occurs in the metabolic fuel type being metabolized.

In an aspect, example methods and systems can comprise monitoring a respiratory quotient of a user during an initiated activity performed by the user. A proportion of a first metabolic fuel type metabolized can be determined during the initiated activity and/or for an additional duration after the initiated activity. The proportion of the first metabolic fuel type being metabolized during the activity can be based on the respiratory quotient. The first metabolic fuel type can be a carbohydrate or a fat, for example. At least one user parameter (e.g., a ventilation rate, a ventilation volume, a heart rate, a weight, a gender, a height, and/or the like) can be received during the duration of the initiated activity. A first amount of the first metabolic fuel type metabolized can be determined based on the duration of the initiated activity, the respiratory quotient, and the at least one user parameter. The first amount of the first metabolic fuel type metabolized can be indicated to the user.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is a block diagram of an example computer system;

FIG. 2 is a block diagram of a ventilation monitor;

FIG. 3 is a flow chart of an example method;

FIG. 4 is a flow chart of an example method; and

FIG. 5 is a graph illustrating how methods and systems can determine a metabolic fuel primarily metabolized.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference to each various individual and collective combinations and permutation of these may not be explicitly disclosed, each is specifically contemplated and described herein, for all methods and systems. This applies to all aspects of this application including, but not limited to, steps in disclosed methods. Thus, if there area variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Methods and systems for determining a metabolic fuel type (e.g., fat, carbohydrate, and/or protein) that is being metabolized by a user (e.g., subject, animal, and/or the like) at a given time and/or over a duration of time are disclosed. The metabolic fuel type being metabolized can be determined based on one or more substance concentrations being ventilated (e.g., inhaled, exhaled, breathed) by the user. Metabolism of different metabolic fuels can produce different ratios of one or more substance concentrations. Monitoring and measuring substance concentrations during ventilation can be used to determine one or more metabolic fuel types being metabolized. For example, metabolism of fat can produce seven molecules of carbon dioxide (CO₂) for every ten molecules of oxygen (O₂) consumed. On the other hand, metabolism of carbohydrates can produce one molecule of carbon dioxide for every molecule of oxygen consumed. The molecules of carbon dioxide can be exhaled from the user during ventilation. The concentration of carbon dioxide and how the concentration of carbon dioxide changes in proportion to oxygen concentration can be used to determine when primarily fat or primarily carbohydrates or glucose is being metabolized. In an aspect, monitoring the substance concentration exhaled can also be used to determine the amount of a metabolic fuel type being metabolized. A substance concentration ratio can be used to determine a proportion of the metabolic fuel type being metabolized. The substance concentration ratio can be used to determine how to initiate the metabolism of a metabolic fuel type based on one or more user parameters heart rate, breath rate, age, gender, weight, and/or the like).

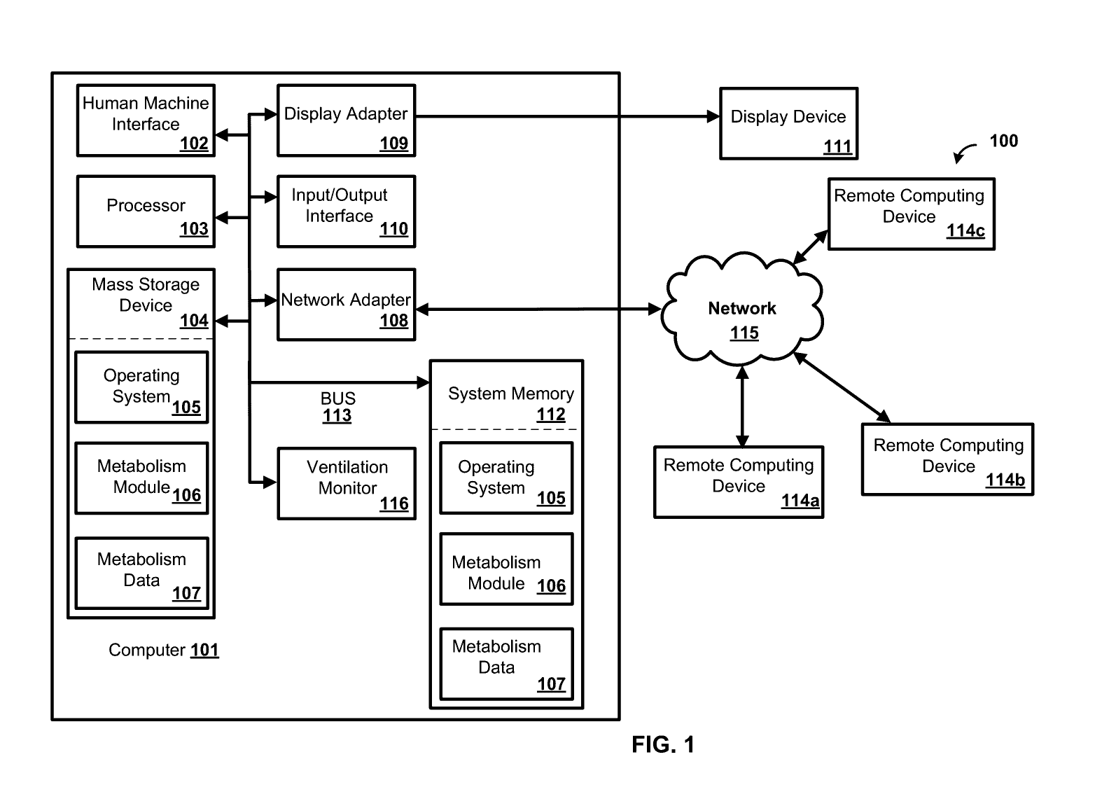

In an aspect, the methods and systems can be implemented on a computer 101 as illustrated in FIG. 1 and described below. Similarly, the methods and systems disclosed can utilize one or more computers to perform one or more functions in one or more locations. FIG. 1 is a block diagram illustrating an exemplary operating environment 100 for performing the disclosed methods. This exemplary operating environment 100 is only an example of an operating environment and is not intended to suggest any limitation as to the scope of use or functionality of operating environment architecture. Neither should the operating environment 100 be interpreted as having any dependency or requirement relating to any one or combination of components illustrated in the exemplary operating environment 100.

The present methods and systems can be operational with numerous other general purpose or special purpose computing system environments or configurations. Examples of computing systems, environments, and/or configurations that can be suitable for use with the systems and methods comprise, but are not limited to, personal computers, equipment containing an electronic device (e.g., treadmill, stair-stepper, stationary or non-stationary bicycle, and the like), server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media including memory storage devices.

Further, one skilled in the art will appreciate that the systems and methods disclosed herein can be implemented via a general-purpose computing device in the form of a computer 101. The computer 101 can comprise one or more components, such as one or more processors 103, a system memory 112, and a bus 113 that couples various components of the computer 101 including the one or more processors 103 to the system memory 112. In the case of multiple processors 103, the computer 101 can utilize parallel computing.

The bus 113 can comprise one or more of several possible types of bus structures, such as a memory bus, memory controller, a peripheral bus, an accelerated graphics port, and a processor or local bus using any of a variety of bus architectures. The bus 113, and all buses specified in this description can also be implemented over a wired or wireless network connection and one or more of the components of the computer 101, such as the one or more processors 103, a mass storage device 104, an operating system 105, metabolism module 106, metabolism data 107, a network adapter 108, system memory 112, an Input/Output Interface 110, a display adapter 109, a display device 111, a ventilation monitor 116 and a human machine interface 102, can be contained within one or more remote computing devices 114a,b,c at physically separate locations, connected through buses of this form, in effect implementing a fully distributed system.

The computer 101 comprises a variety of computer readable media. Exemplary readable media can be any available media that is accessible by the computer 101 and comprises, for example and not meant to be limiting, both volatile and non-volatile media, removable and non-removable media. The system memory 112 can comprise computer readable media in the form of volatile memory, such as random access memory (RAM), and/or non-volatile memory, such as read only memory (ROM). The system memory 112 can comprise data such as metabolism data 107 and/or program modules such as operating system 105 and metabolism module 106 that are accessible to and/or are operated on by the one or more processors 103.

In another aspect, the computer 101 can also comprise other removable/non-removable, volatile/non-volatile computer storage media. The mass storage device 104 can provide non-volatile storage of computer code, computer readable instructions, data structures, program modules, and other data for the computer 101. For example, a mass storage device 104 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 104, including by way of example, an operating system 105 and metabolism module 106. One or more of the operating system 105 and metabolism module 106 (or some combination thereof) can comprise elements of program modules and the metabolism module 106. Metabolism data 107 can also be stored on the mass storage device 104. Metabolism data 107 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple locations within the network 115.

In another aspect, the user can enter commands and information into the computer 101 via an input device (not shown). Examples of such input devices comprise, but are not limited to, a keyboard, pointing device (e.g., a computer mouse, remote control), a microphone, a joystick, a scanner, tactile input devices such as gloves, and other body coverings, motion sensor, device incorporated into exercise equipment, and the like. These and other input devices can be connected to the one or more processors 103 via a human machine interface 102 that is coupled to the bus 113, but can be connected by other interface and bus structures, such as a parallel port, game port, an IEEE 1394 Port (also known as a Firewire port), a serial port, network adapter 108, and/or a universal serial bus (USB).

In yet another aspect, a display device 111 can also be connected to the bus 113 via an interface, such as a display adapter 109. It is contemplated that the computer 101 can have more than one display adapter 109 and the computer 101 can have more than one display device 111. For example, a display device 111 can be a monitor, an LCD (Liquid Crystal Display), light emitting diode (LED) display, television, smart lens, smart glass, and/ or a projector. In addition to the display device 111, other output peripheral devices can comprise components such as speakers (not shown) and a printer (no(shown), which can be connected to the computer 101 via Input/Output Interface 110. Any step and/or result of the methods can be output in any form to an output device. Such output can be any form of visual representation, including, but not limited to, textual, graphical, animation, audio, tactile, and the like. The display device 111 and computer 101 can be part of one device, or separate devices.

The computer 101 can operate in a networked environment using logical connections to one or more remote computing devices 114a,b,c. By way of example, a remote computing device 114a,b,c can be a personal computer, computing station (e.g., workstation), portable computer (e.g., laptop, mobile phone, tablet device), smart device (e.g., smartphone, smart watch, activity (racker, smart apparel, smart accessory), security and/or monitoring device, device incorporated into exercise equipment, a server, a router, a network computer, a peer device, edge device or other common network node, and so on. Logical connections between the computer 101 and a remote computing device 114a,b,c can be made via a network 115, such as a local area network (LAN) and/or a general wide area network (WAN). Such network connections can be through a network adapter 108. A network adapter 108 can be implemented in both wired and wireless environments. Such networking environments are conventional and commonplace in dwellings, offices, gyms, enterprise-wide computer networks, intranets, and the Internet.

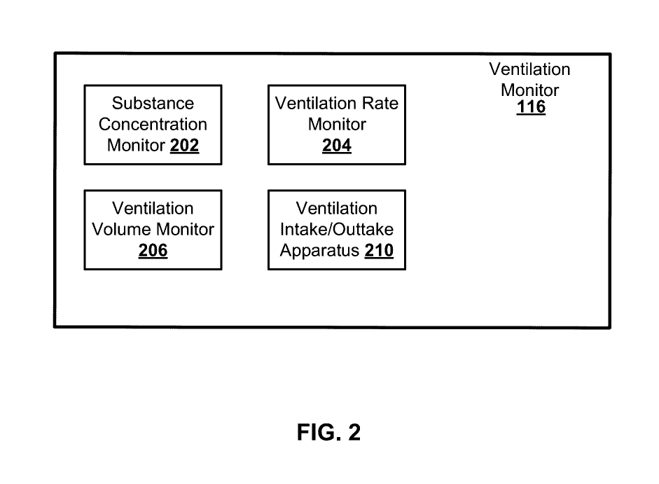

In yet another aspect, one or more physiological monitors such as a ventilation monitor 116 can be connected to the bus 113. In an aspect, the ventilation monitor 116 can be part of the computer 101. In another aspect the ventilation monitor 116 can be a peripheral device that can be connected to the computer system via the Input/Output Interface 110. In another aspect, the ventilation monitor 116 can be a remote computing device 114a,b,c and communicate to the computer 101 via the network 115. In an example, the ventilation monitor 116 can be a computing device that a user wears over or near the user's mouth and/or nose to monitor, measure, calculate, determine, and/or the like one or more physiological parameters such as one or more substance concentrations that the user is ventilating, rate at which the user is ventilating, volume of ventilation, and/or the like. Physiological parameters can be transferred and/or stored as metabolism data 107 in computer 101. The metabolism data 107 can be used by the metabolism module 106 to perform methods described here in. Components of the ventilation monitor 116 are further explained in FIG. 2.

In an aspect, additional physiological monitors can be used to monitor, measure, calculate, determine, and/or the like one or more physiological parameters to be used in conjunction with the physiological parameters of the ventilation monitor 116 as metabolism data 107. In various aspects, the additional physiological monitors can be part of the computer 101, a remote computing device 114a,b,c, or peripheral device. In an aspect, the additional physiological monitors can comprise a heart rate monitor, a blood pressure monitor, a blood glucose monitor, a thermometer, and/or the like.

In an aspect, the physiological monitors can communicate with the computer device by a short-range wireless protocol. For example, the short-range wireless protocols can be Bluetooth®, Bluetooth Low Energy protocols, infrared data association (IrDA), ANT, Zigbee, near field communications (NEC) and the like. The wireless connection between the physiological monitor such as the ventilation monitor 116 can allow a user more mobility when performing various movements without having to be restricted to carrying extra devices or limited to a location of the computer 101.

For purposes of illustration, application programs and other executable program components such as the operating system 105 are illustrated herein as discrete blocks, although it is recognized that such programs and components can reside at various times in different storage components of the computer 101, and are executed by the one or more processors 103 of the computer 101. An implementation of the metabolism module 106 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any method or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

FIG. 2 illustrates a block diagram of components of a ventilation monitor 116 of FIG. 1. In an aspect the ventilation monitor 116 can include but is not limited to a substance concentration monitor 202. In an aspect, the ventilation monitor 116 can also include but is not limited to a ventilation rate monitor 204 and a ventilation volume monitor 206 (e.g., spirometer). The ventilation monitor 116 can also include a ventilation intake/outtake apparatus 210. A user can exhale into or near the ventilation intake/outtake apparatus 210. As air is being transferred through the ventilation intake/outtake apparatus 210, the various monitors 202, 204, and 206 can be taking measurements of the air passing through the ventilation monitor 116, and the ventilation intake/outtake apparatus 210. In an aspect, the substance concentration monitor 202 can determine (e.g., measure, calculate) substance concentrations and necessary ratios in each ventilation. The substance concentration monitor 202 can have one or more sensors that can detect the amount and/or concentration of one or more respective substances. For example, the substance concentration monitor 202 can have a sensor that can determine the amount and/or concentration of carbon dioxide during ventilation. In addition, the substance concentration monitor 202 can determine the amount and/or concentration of oxygen consumed for each ventilation. In another contemplated aspect, other sensors that detect substances can help determine a proportion of metabolic fuel type being metabolized.

In an aspect, the ventilation monitor 116 can include a ventilation rate monitor 204 which can determine (e.g., measure, calculate, and the like) the rate at which a user is ventilating at in terms of ventilations per minute. The ventilation rate monitor 204 can also determine when a user is exhaling and inhaling through the ventilation monitor 116 In an aspect, the ventilation volume monitor 206 can be used to determine (e.g., measure, calculate, and the like) the volume of air inhaled and exhaled for each ventilation. In an aspect, metabolism data 107 collected from the substance concentration monitor 202, the ventilation rate monitor 204, and the volume monitor 206 can be associated with each other by a time stamp of when the measurement of each monitor 202, 204, 206 was taken. The time stamp can be used to indicate a time of interest in the monitoring of the metabolism data 107. For example, the time of interest can be when RQ rises from rest to exercise, when RQ falls from initial exercise values indicating a metabolic switch point to fat metabolism, and the period after exercise when RQ is below at rest RQ before initiating exercise. The duration of post-exercise indicated by the time stamp can be used to determine the amount of fat burned after exercise.

Referring again to FIG. 1, the metabolism data 107 measured by the ventilation monitor 116 can be stored in the mass storage device 104 and/or system memory 112. In an aspect, the metabolism module 106 can use the metabolism data 107 to determine a proportion of metabolic fuel types a user is metabolizing. The metabolism module 106 can determine the proportion of the metabolic fuel types based on the concentration of one or more substances being ventilated. The metabolism module 106 can receive a measurement of a substance concentration (e.g., oxygen, nitrogen carbon dioxide, water vapor, and/or the like) exhaled by a user during a time period. The substance concentration depends on a metabolic fuel type metabolized or the proportion of the metabolic fuel types being metabolized by the user. In an aspect, the metabolism module 106 can determine a time of interest when a first metabolic fuel type is being primarily metabolized by the user based on the measurements of substance concentrations. The metabolism module 106 can determine that a ratio of the substance concentrations indicates a first metabolic fuel type is being primarily metabolized at the time of interest. Furthermore, the metabolism module 106 can determine that the ratio of the substance concentrations indicates a second metabolic fuel is being primarily metabolized by the user at a time of interest. In an aspect, both the first metabolic fuel type and the second metabolic fuel type can be metabolized simultaneously. The metabolism module 106 can indicate a metabolic switch point to the user. The metabolic switch point can be when one of the metabolic fuel types switches to being primarily metabolized over the other(s). In an aspect, the metabolism module 106 can indicate to the user when the proportion in metabolic fuel types being metabolized changes. In an aspect, the metabolism module can determine the amount of each metabolic fuel type that has been metabolized based on one or more substance concentrations and one or more physiological parameters of the user.

For example, the metabolism module 106 can indicate when exercise starts, the metabolic switch point when fat burning is initiated by appropriate exercise, and the duration after the end of exercise when fat burning persists. As another example, the metabolism module 106 can determine a proportion of metabolic fuels types (e.g. protein, carbohydrate, fat, and the like) being metabolized by a user based on a substance concentration ratio of carbon dioxide produced to oxygen consumed by the user. As an example, the metabolism module 106 can determine a carbohydrate amount and a fat amount being metabolized by the user.

During metabolism, biochemical reactions occur that convert metabolic fuels into one or more forms of energy to be used to produce body movement. During these processes, chemical oxidation reactions occur in which oxygen is consumed and carbon dioxide is produced as a waste product. These processes require the transport of oxygen from the air to cells of a user and the transport of carbon dioxide from the cells to the air. Oxygen is received through lungs when the user inhales and enters the bloodstream to be received by the cells of the user for metabolism of a metabolic fuel. Carbon dioxide, a waste product of metabolism, is carried by the blood to the lungs, and is expelled from the user through the lungs when the user exhales. Ventilation is a part of respiration where the user inhales oxygen and exhales carbon dioxide.

At rest, metabolism can be fueled by a mixture of fuel types, primarily carbohydrate (glucose) and fat, with some protein depending on diet. At rest, the o of carbon dioxide produced as a function of oxygen consumed reflects this mixture of fuel types with a respiratory exchange ratio (Respiratory Quotient; RQ) between 0.7 and 1.0 (0.7 being the RQ produced when fat is used as a fuel, and 1.0 being the RQ associated with pure glucose metabolism).

With the onset of exercise, metabolic demands can increase, and these increased demands can be initially satisfied by metabolism of glucose derived from blood glucose or carbohydrate stored in the liver as either glucose or glycogen. Total metabolism can increase, and the increase from resting conditions can be fueled by glucose. So the RQ at the beginning of exercise can reflect a metabolism dominated by glucose as a fuel. During exercise, total metabolic rate can increase, and the RQ can rise toward 1.0. The greater the intensity of the exercise, the more glucose metabolism dominates and the RQ will get closer to 1.0. Under all conditions, this early phase can be characterized by an increase RQ with the onset of exercise.

Some levels of exercise can be totally fueled by glucose metabolism. However, if the intensity of the exercise is appropriate, a metabolic switch point can be reached. The metabolic switch point can indicate an increased use of stored fat as a fuel. The proportion of metabolism fueled by fat will increase and the proportion fueled by glucose will decline. As such, when fat stores begin to be utilized, the RQ will fall from 1.0 toward 0.7. The exercise intensity necessary to induce this metabolic adjustment can be complex. Too little exercise, and metabolism never switches to fat as a fuel. Too intense exercise can also preferentially use only glucose. Just the right amount of moderate intensity exercise, for just the right amount of time, is required to induce the metabolic recruitment of stored fat.

This metabolic switch point can differ between individuals based on the user's age, biochemistry, diet, fitness, combinations thereof, and the like. Once initiated, fat metabolism can continue to be maintained for the duration of exercise, and can continue for a time beyond the period of exercise. After exercise is stopped, metabolic adjustment induced by exercise can result in a larger proportion of resting metabolism being fueled by stored fat than was the case at rest before exercise. In many situations, maintenance of these processes can result in a situation where the amount of stored fat burned to support resting metabolism after exercise can be greater than the stored fat burned during the exercise itself. If a user is using exercise to lose weight, the most beneficial strategy can be to get to the metabolic switch point as efficiently as possible, so all subsequent exercise and a period of subsequent rest will burn off stored fat.

The RQ can fluctuate based on what a user eats, when the user eats, level of activity the user is performing, and the like. While a user is at rest, the RQ is usually an intermediate value between 0.7 and 1.0 indicating a mixed metabolism of fats, proteins and carbohydrates. At rest RQ would be expected to approximate 0.85 as an estimate. As a user increases levels of physical activity such as during exercise, metabolic fuel requirements increase and these increased requirements are almost exclusively met by an increase in carbohydrate metabolism. As total metabolism is increased, and the proportion of that metabolism met by carbohydrate fuels is increased, RQ rises from the resting value (0.85) toward the value expected during pure carbohydrate metabolism (1.0). Under certain circumstances, determined by factors such as exercise intensity and duration, and a user's age, diet and fitness level, a metabolic change (e.g. metabolic switch point) will occur that mobilizes stored fat as a fuel for metabolism.

As metabolism switches to incorporate fat as a fuel during the period of exercise, RQ decreases towards the value expected during pure fat metabolism (0.7). As such the point where RQ falls during a period of exercise indicates that metabolic adjustments have been initiated that mobilize stored fat as a metabolic fuel. The degree to which RQ falls can be used to indicate the proportion of metabolism fueled by “burning fat.” As a user decreases levels of physical activity at the end of a period of exercise, metabolic fuel requirements return to resting levels. Where metabolic adjustments that mobilize stored fat not initiated during exercise, RQ would return to resting values as total metabolism falls and the proportion of that metabolism met by carbohydrate fuels returns to resting levels (from exercise RQ values approximating 1.0 toward resting values estimated at 0.85). In cases where metabolic adjustments that mobilize stored fat were initiated during exercise, these adjustments persist for a period following the end of the period of exercise. In such cases, RQ values fall below pre-exercise values (below 0.85) illustrating that post-exercise metabolism continues to incorporate fat as a fuel. The degree and duration that RQ is maintained below pre-exercise values indicates the proportion of post-exercise metabolism fueled by “burning fat” after exercise. The RQ can help the user determine the metabolic switch point during a particular activity that is sufficient to induce increased fat metabolism, optimal activity level to maximize fat metabolism and the degree and duration that fat metabolism persists following a particular activity. Determining which metabolic fuel is being primarily metabolized and when the metabolic fuel is being primarily metabolized can be helpful in many situations such as in athletics for athletes to monitor performance, dieters seeking weight loss, and the like.

Returning to FIG, 1, in an aspect, the metabolism module 106 can use the metabolism data 107 to calculate RQ of a user of the device. Using the metabolism data 107, the metabolism module 106 can calculate an amount of oxygen that is consumed by the user by determining the amount of oxygen being inhaled and subtracting the amount of oxygen being exhaled by the user. The amount of oxygen can be determined based on the concentration of oxygen and the volume of air during ventilation. Likewise, the amount of carbon dioxide produced can be the difference of the amount of carbon dioxide inhaled and the amount of carbon dioxide exhaled. The amount of carbon dioxide can be based on the concentration of the carbon dioxide and volume of air during ventilation.

In an aspect, the computer 101 can indicate to the user the RQ so the user knows which metabolic fuel is being metabolized. In another aspect, the computer 101 can indicate to the user when a change in degree to which a particular metabolic fuel is being metabolized. As an example, the computer 101 can indicate to the user that the user has begun to initiate fat metabolism when the RQ begins to fall from 1.0 toward 0.7 during an activity.

In an aspect, the computer 101 can gather metabolism data 107 other than from a ventilation monitor. As an example, the computer 101 can gather heart rate data and other physiological parameters of the user such as an age, a weight, a temperature, a gender, a height, a muscle to fat ratio, and/or the like. The metabolism module 106 can use at least a portion of the metabolism data 107 and the RQ for given time periods to estimate an amount of calories metabolized from each fuel type.

In an aspect, the metabolism module 106 can provide recommendations to initiate metabolism of a particular metabolic fuel. The metabolism module 106 can leant based on past results of the user, what activities were performed, intensity of activities, and length of time performing the activity, combinations thereof, and the like that can lead to a targeted RQ level. For example, previous use of the device can identify more optimal activity patterns that induced primarily fat metabolism in the specific activity. The optimal activity patterns may depend on factors such as a user's age, weight, diet and fitness. The metabolism module 106 may use this information to “coach” the user to adjust current activity to target that which was identified as optimal on prior occasions. This coaching could motivate the user to increase the intensity or duration of an activity, or may suggest a decrease in intensity, to optimize fat metabolism both during and following the period of activity.

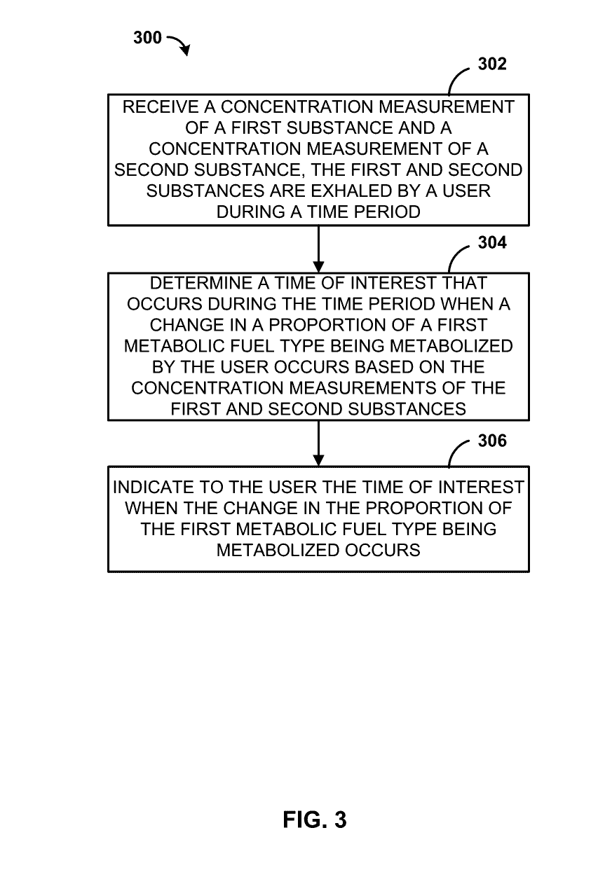

FIG. 3 illustrates a method 300 of determining a metabolic fuel type being metabolized. In step 302, a computer, such as computer 101 of FIG. 1, can receive a concentration measurement of a first substance and a concentration measurement of a second substance(e.g., oxygen, carbon dioxide, water vapor and/or the like). The first substance and the second substance are exhaled by a user during a time period. Concentrations of the first substance and the second substance depend on a proportion of metabolic fuel types being metabolized by the user. As an example, a concentration of carbon dioxide exhaled by the user can be less when a fat metabolic fuel is being metabolized than when a carbohydrate metabolic fuel is being metabolized.

In step 304, the computer 101 can determine a time of interest that occurs during the time period when a change in the proportion of the metabolic fuel types being metabolized by the user occurs. In an aspect, the computer 101 can determine a ratio of the concentration measurement of the first substance and the concentration measurement of the second substance indicates a first metabolic fuel is being primarily metabolized at the time of interest. Furthermore, the computer 101 can determine the ratio of the concentrations of the first substance and the second substance indicates a second metabolic fuel is being primarily metabolized by the user at the time of interest. For instance, the computer 101 can determine from the ratio carbon dioxide concentration and oxygen concentration exhaled from the user can indicate primarily that carbohydrates are being metabolized at the time of interest. In another aspect, the computer 101 can determine the relative proportion of carbon dioxide concentration to oxygen concentration indicates that fat molecules are being used as the primary metabolic fuel. In an aspect, the computer 101 can determine the relative proportion of carbon dioxide concentration to oxygen concentration indicates that a change in the proportion of fat molecules to carbohydrates molecules occurred. In an aspect, the computer 101 can determine a current RQ of the user based on measurements from a ventilation monitor 116. The ventilation monitor 116 can be configured to measure a volume of ventilation, a substance concentration, a ventilation rate, and/or the like.

In step 306, the computer can indicate to the user the time of interest when the change in the proportion of the metabolic fuel type being metabolized occurs. In an aspect, the computer 101 can indicate to the user the mixture/proportion of metabolic fuel types being metabolized by a user and the increases in metabolic fuels used during exercise. In an aspect, the computer 101 can indicate to the user through the user interface a metabolic switch point when the user has begun to increase the use of fat as a primary metabolic fuel. The computer 101 can visually indicate to the user through the visual display, or any other sensory feedback mechanism such as a light or video display, an audio indication, a vibration indication, and/or the like.

In an aspect, the computer 101 can provide recommendations to initiate metabolism of a particular metabolic fuel. The metabolism module 106 can learn based on past results of the user, what activities were performed, intensity of activities, and length of time performing the activity, combinations thereof, and the like that can lead to a targeted RQ level. For example, previous use of the device can identify more optimal activity patterns that induced primarily fat metabolism in the specific activity. The optimal activity patterns may depend on factors such as a user's age, a weight, a diet, and a fitness level. The metabolism module 106 may use this information to “coach” the user to adjust current activity to target that which was identified as optimal on prior occasions. This coaching could motivate the user to increase the intensity or duration of an activity, or may suggest a decrease in intensity, to optimize fat metabolism both during and following the period of activity.

In an aspect, the computer 101 can provide recommendations to initiate metabolism of a particular metabolic fuel by receiving a measurement of at least one user parameter for the time period such as a heart rate, for example. The computer 101 can also receive a targeted time during the time period when the first metabolic fuel type should begin being primarily metabolized. The computer 101 can correlate the measurement of the at least one user parameter with the time of interest. The computer can then determine whether the targeted time is substantially similar to the time of interest. When the time of interest is not substantially similar to the targeted time, the computer 101 can provide a recommendation to the user on how to adjust the at least one user parameter so that the time of interest is substantially similar to the targeted time when the activity is performed again. For example, the heart rate of the user, based on past stored exercise sessions, may need to be increased to reach a desired level of fat metabolism. The computer 101 could suggest increasing/decreasing speed, change in resistance, incline, combinations thereof, and the like on a particular piece of exercise equipment. Compiling results of various past exercise sessions for a user, the computer 101 can provide recommendations before an exercise session as to what may be the most efficient exercise to reach a desired level of metabolism of a particular fuel. The computer 101 can use other factors besides user parameters such as environmental factors (e.g., a time of day, a temperature, an air pressure, a wind speed, combinations thereof, and the like) when determining a recommendation to initiate primary metabolism of a particular metabolic fuel.

The methods and systems can employ artificial intelligence (AI) techniques such as machine learning and iterative learning. Examples of such techniques include, but are not limited to, expert systems, case based reasoning, Bayesian networks, behavior based Al, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

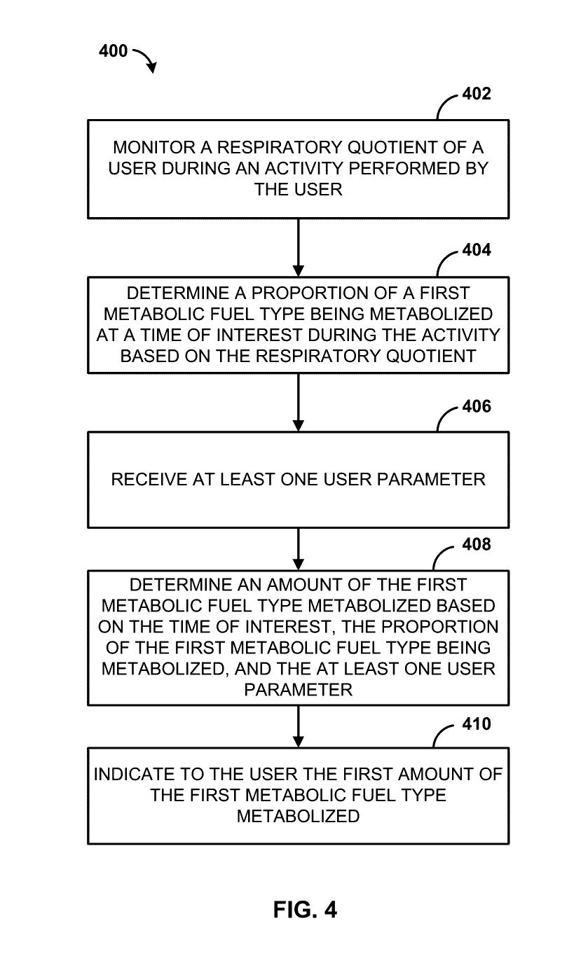

FIG. 4 illustrates a method 400 of determining a proportion of metabolic fuel types being metabolized. In step 402, a computer 101 can monitor a respiratory quotient (RQ) of a user during an activity performed by the user. In an aspect, the RQ can be a ratio of a first substance concentration to a second substance concentration where the first substance concentration changes based on the type of metabolic fuel being metabolized by the user. In an aspect, the first substance can be carbon dioxide and the second substance can be oxygen. The RQ can be the ratio of the carbon dioxide produced by metabolism to the amount of oxygen consumed by the user. The amount of carbon dioxide produced can be the difference between the amount of carbon dioxide exhaled and the amount of carbon dioxide inhaled during ventilation. The amount of oxygen consumed can be the difference between the amount of oxygen inhaled to the amount of oxygen exhaled during ventilation.

In step 404, the computer 101 can determine a proportion of a first metabolic fuel type being metabolized during the activity based on the RQ. A predetermined pattern of change in RQ, indicating a change in use of one or more metabolic fuel types, can be stored in the computer 101. The computer 101 can continuously and/or at intervals determine the RQ of the user. The computer 101 can compare the RQ to the previous and/or the predetermined pattern of change in RQ to determine a change in proportion of which metabolic fuel types are metabolized. In an aspect, the computer 101 can determine the metabolic fuel types being primarily metabolized based on the RQ. In an aspect, the computer 101 can determine a change in the proportion of the first metabolic fuel type being metabolized during the activity and/or after the activity. In an aspect, the computer 101 can monitor a time of interest at which the first metabolic fuel type is being primarily metabolized. The computer 101 can then determine a time of interest, such as a metabolic switch point, when a change in the metabolic fuel type being primarily metabolized occurs based on a change in the RQ. Again, the computer system 101 can compare the RQ to the previous or predetermined patterns of change in RQ stored on the computer system 101 or can detect a change in the RQ which indicates that a second metabolic fuel type has begun to metabolize at a greater degree. The computer 101 can determine from monitoring the RQ, a proportion of the second metabolic fuel type being metabolized during the activity and/or after the activity.

In step 406, the computer 101 can receive at least one user parameter. A user parameter can comprise one or more of a ventilation rate, a ventilation volume, a heart rate, a temperature level, a blood pressure level, a blood sugar level, and/or the like. In an aspect, the user parameter can comprise a static user parameter such as one or more of an age, a gender, a weight, a height, and/or the like. The at least one user parameter can be stored in the computer 101 as metabolism data 107. The at least one user parameter can be measured by one or more sensors, monitors, and/or the like that can be in communication with the computer 101, comprise the computer 101, and/or comprise the ventilation monitor 116, and/or the like. In an aspect, the at least one user parameter can be inputted to the computer 101 by the user through the human machine interface 102.

In step 408, a first amount of the first metabolic fuel type metabolized can be determined by the computer 101 based on the time of interest and the at least one user parameter. The time of interest can be at least the duration of the activity. In an aspect, the computer 101 can use the concentrations of the first substance and the second substance to determine the RQ and a user parameter of ventilation volume to determine the amount of the first substance and second substance per ventilation. The computer 101 can sum up the amount of the first substance and second substance per ventilation at the time of interest to arrive at a total amount of the first substance and second substance produced. Based on the total amount of the first substance produced, and the second substance produced, the computer 101 can determine a proportion of the first metabolic fuel metabolized based on the chemical processes used to metabolize the first metabolic fuel. Likewise, the computer 101 can determine an amount of the second metabolic fuel type metabolized based on the time of interest and at least one user parameter. In an aspect, the computer 101 can convert the amount of the first metabolic fuel type and the amount of the second metabolic fuel type into total calories metabolized during the activity.

In step 410, the computer 101 can indicate to the user the first amount of the first metabolic fuel type metabolized. The computer 101 can indicate to the user through the user interface when the user has begun to primarily metabolize the first metabolic fuel. The computer 101 can visually indicate to the user through the visual display, or any other sensory feedback mechanism such as an audio indication, a vibration indication, and/or the like. Furthermore, the computer 101 can indicate to the user the second amount of the second metabolic fuel type metabolized. The computer 101 can indicate to the user through the user interface when the user begins to primarily metabolize the second metabolic fuel.

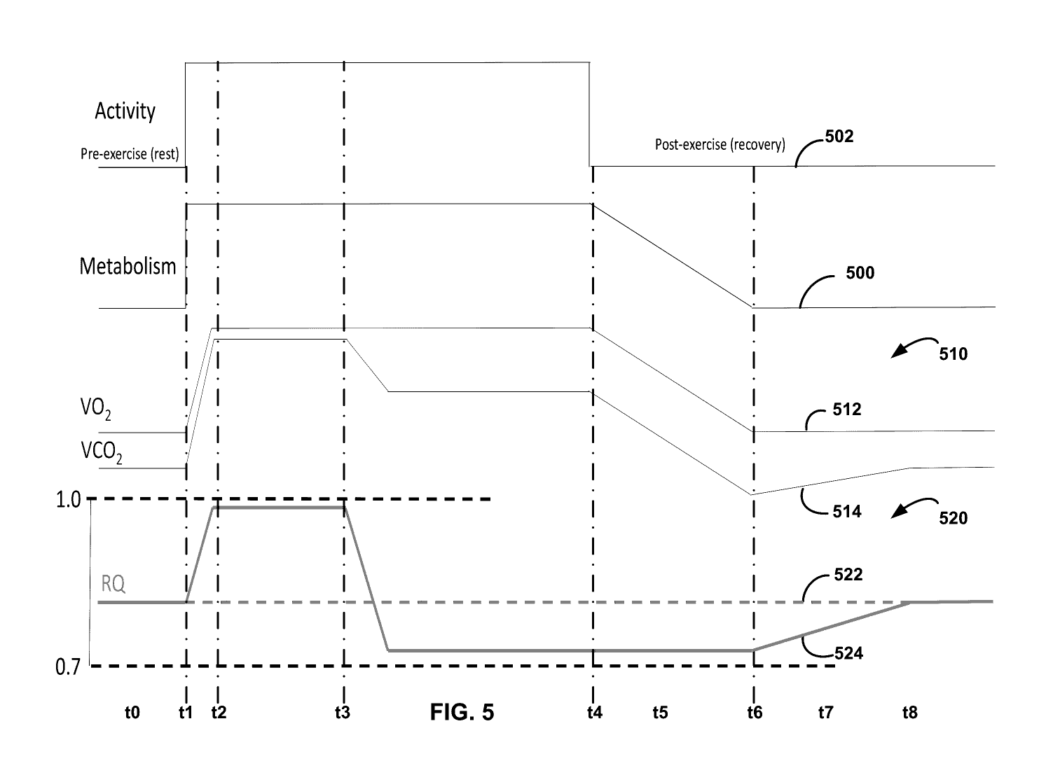

FIG. 5 illustrates a graphical representation for determining a proportion of a metabolic fuel type being metabolized during an activity. In an aspect, graph 500 illustrates the level of metabolism over a time duration before, during, and following a period of exercise represented by graph 502. For example, graph 500 illustrates the metabolism level while a user is performing the activity of rest, exercise, and recovering from exercise. Graph 510 illustrates the volume of a first substance inhaled in relation to a second substance exhaled over the same time duration as the level of metabolism changes. For example, graph 510 illustrates the amount of oxygen consumed for the time duration (the volume of oxygen consumed is designated VO₂) at line 512 and an amount of carbon dioxide exhaled (the volume of carbon dioxide produced is designated VCO₂) at line 514 during the time duration. Graph 520 illustrates the ratio of the second substance exhaled to the first substance consumed during the time duration of the level of metabolism. For example, graph 520 illustrates the respiratory quotient (RQ) during the time duration. Line 522 represents the RQ at rest and line 524 represents the RQ over the time duration as the level of metabolism changes.

For example, at time t0, the user can be at rest as illustrated by graph 500. When the metabolic fuel of a fat is metabolized, for every ten molecules of oxygen that are consumed there are seven molecules of carbon dioxide released. When a carbohydrate is metabolized, one molecule of carbon dioxide is released for every molecule of oxygen consumed. While at rest, the user can be metabolizing more than one fuel type. For example, the user can be metabolizing both fats and carbohydrates. While at rest, the user can be exhaling lesser amounts of carbon dioxide than the amount of oxygen the user is consuming. Since the user is metabolizing both fats and carbohydrates the amount of carbon dioxide exhaled is going to be less than the oxygen consumed due to the 7:10 ratio of carbon dioxide to oxygen consumed when metabolizing a fat. In graph 510, at t0 line 512 illustrates the amount of oxygen consumed and line 514 illustrates the amount of carbon dioxide released, which is less than line 512. In graph 520, at time t0 the line 522 representing RQ is the same as the line 524 representing the RQ at rest. Since more than one fuel type is being metabolized, the line 522 falls between 1.0 and 0.7 which represents metabolizing only carbohydrates and only fats, respectively.

At time t1, the user of the system can begin an exercise or some sort of activity. At time t1, the level of metabolism goes from rest to exercise at graph 502 and graph 500. In graph 510, the amount of oxygen consumed begins to increase as illustrated at line 512. The amount of carbon dioxide produced also begins increasing at time t1 as indicated by line 514. The difference between the amount of oxygen and the amount of carbon dioxide begins to decrease until the lines 512 and 514 almost converge at time t2. This near convergence represents the increase in the metabolism of the carbohydrate fuel type in comparison to the fat fuel type. This is also illustrated by graph 520, where at time a the RQ of the user begins to rise, which indicates an increase in the amount of carbohydrates being metabolized with respect to the amount of fat being metabolized.

At time t2, in graph 500 the user is still exercising. In graph 510, line 512 is approximately the same as the volume of carbon dioxide exhaled in line 514. In graph 520, the RQ in line 524 has risen to approximately 1.0, which indicates primarily carbohydrates are being metabolized.

At time t3 in graph 500, the user is still exercising as illustrated by graph 502. In graph 510, the amount of amount of carbon dioxide in line 514 is beginning to decrease as the amount of oxygen consumed in line 512 remains constant. This indicates that more fat is being metabolized since less carbon dioxide is produced with respect to oxygen consumed. In graph 520, the RQ in line 524 begins to decrease from approximately 1.0, which confirms a metabolic switch point where a metabolic adjustment is being initiated to initiate greater mobilization of fat as a fuel for metabolism.

At time t4, in graph 502 the user has just finished exercising or has begun a less vigorous exercise. Therefore, the metabolism of the user begins to decrease to resting state in graph 500. In graph 510, the amount of oxygen consumed in line 512 and the amount of carbon dioxide in line 514 decrease as metabolism falls. This decrease indicates that during the period of recovery when metabolism is falling more fat is being metabolized with respect to the amount of carbohydrates being metabolized than at time t3. In graph 520, the RQ of line 524 is still approximating 0.7 which indicates that elevated levels of fat are continuing to be metabolized compared to carbohydrates, during the post-exercise period.

At time t5 (the period between t4 and t6), in graph 502 the user continues to recover from the exercise and metabolism decreases as illustrated by graph 500. In graph 510, the volume of oxygen consumed fails in proportion to the fall in metabolism as indicated by lines 512 and graph 500 while the volume of carbon dioxide released also falls in proportion to metabolism. However, the ratio of carbon dioxide produced as a function of oxygen consumed remains constant as indicated by line 514. The difference in the volume of carbon dioxide released and the volume of oxygen consumed is the greatest through time t5. In graph 520, the RQ has decreased to nearly 0.7 as illustrated by line 524. This indicates that fat is being metabolized to the greatest degree by the user through time t5. During this period the difference in the volumes remains approximately the same. The difference indicates that fat is still being metabolized over carbohydrates even after the user has stopped exercising.

At time to, in graph 502, the user is now at rest and the level of metabolism has decreased to resting metabolism, as illustrated by graph 500, such that the user has a resting heart rate and a resting breathing rate. In graph 510, the volume of oxygen consumed illustrated by line 512 is constant and the volume of carbon dioxide released illustrated by line 514 begins to increase. This illustrates that a balance of fuel types being used for metabolism is beginning to return to metabolism that occurs at rest, specifically the metabolic adjustment that initiated the greater degree of fat metabolism during the period of exercise (time t3) is beginning to reverse. In graph 520, RQ is still close to 0.7 at time t6 as illustrated by line 524 but begins to rise towards at rest RQ illustrated by line 522.

At time t7, in graph 502 the user is at rest and the metabolism of the user is at rest as is illustrated by graph 500. In graph 510, the difference between the volume of oxygen consumed and the volume of carbon dioxide released begins to decrease as indicated by the constant oxygen volume at line 512 and the increasing carbon dioxide volume at line 514. In graph 520, the RQ is still rising towards at rest RQ. At time t8, the graphs 500, 502, 510, and 520 have returned to their original at rest levels of time t0. This indicates both that overall metabolism is at resting values, and that the balance of metabolic fuels has returned to that occurring before exercise.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method, comprising:
receiving a concentration measurement of a first substance and a concentration measurement of a second substance, wherein the first substance and the second substance are exhaled by a user during a time period;
determining a time of interest that occurs during the time period when a change in a proportion of a first metabolic fuel type being metabolized by the user occurs based on the concentration measurement of the first substance and the concentration measurement of the second substance; and
indicating to the user the time of interest when the change in the proportion of the first metabolic fuel type being metabolized occurs.

2. The method of claim 1 wherein the first substance is carbon dioxide and the second substance is oxygen.

3. The method of claim 1, wherein determining a time of interest that occurs during the time period when the change in the proportion of the first metabolic fuel type being metabolized occurs by the user based on the concentration measurement of the first substance and the concentration measurement of the second substance comprises:
determining a respiratory quotient based on a ratio of a carbon dioxide concentration measurement and an oxygen concentration measurement at the time of interest;
determining the proportion of the first metabolic fuel type being metabolized by the user based on the respiratory quotient; and
determining the change in the proportion of the first metabolic fuel type being metabolized based on the proportion of the first metabolic fuel type being metabolized by the user.

4. The method of claim 1, further comprising:
receiving a measurement of at least one user parameter for the time period;
determining a first rate at which the first metabolic fuel type is being metabolized based on the measurement of the at least one user parameter, the concentration measurement of the first substance and the concentration measurement of the second substance; and
indicating to the user the first rate at which the first metabolic fuel type is being metabolized.

5. The method of claim 4, further comprising:
determining an amount of the first metabolic fuel type metabolized during the time period based on the first rate; and
indicating to the user the amount of the first metabolic fuel type metabolized.

6. The method of claim 5, further comprising:
determining a second rate at which a second metabolic fuel type is being metabolized based on the measurement of the at least one user parameter, the concentration measurement of the first substance, and the concentration measurement of the second substance; and
determining an amount of the second metabolic fuel type metabolized based on the second rate.

7. The method of claim 6, wherein the first metabolic fuel type is a fat and the second metabolic fuel type is a carbohydrate.

8. The method of claim 6, further comprising, determining a calorie amount metabolized by the user based on the amount of the first metabolic fuel type and the amount of the second metabolic fuel type.

9. The method of claim 1, further comprising:
receiving a measurement of at least one user parameter for the time period;
receiving a targeted time during the time period when the first metabolic fuel type begins being primarily metabolized;
correlating the measurement of the at least one user parameter the time of interest;
determining whether the targeted time is substantially similar to the time of interest; and
when the time of interest is not substantially similar to the targeted time, providing a recommendation to the user on how to adjust the at least one user parameter so that the time of interest is substantially similar to the targeted time.

10. A method, comprising:
monitoring a respiratory quotient of a user during an activity performed by the user;
determining a proportion of a first metabolic fuel type being metabolized at a time of interest during the activity based on the respiratory quotient;
receiving at least one user parameter;
determining an amount of the first metabolic fuel type metabolized based on a time of interest, the proportion of the first metabolic fuel type being metabolized, and the at least one user parameter; and
indicating to the user the amount of the first metabolic fuel type metabolized.

11. The method of claim 10, further comprising:
determining a proportion of a second metabolic fuel type being metabolized during the activity based on the respiratory quotient;
determining an amount of the second metabolic fuel type metabolized based on the time of interest, the proportion of the second metabolic fuel type being metabolized, and the at least one user parameter; and
indicating to the user the amount of the second metabolic fuel type metabolized.

12. The method of claim 11, further comprising, determining a recommendation to alter the at least one user parameter so that the proportion of the first metabolic fuel type to the second metabolic fuel type is altered.

13. The method of claim 11, wherein the first metabolic fuel type is a fat and the second metabolic fuel type is a carbohydrate.

14. The method of claim 10, wherein the respiratory quotient is based on a ratio of carbon dioxide and oxygen expired in a breath of the user.

15. The method of claim 10, wherein the user parameter is at least one of a ventilation rate, a ventilation volume, a heart rate, a temperature, a blood pressure, and a blood sugar level.

16. The method of claim 10, wherein determining the amount of the first metabolic fuel type metabolized is based on a static user parameter.

17. The method of claim 16, wherein the static user parameter is at least one of an age, a gender, a height, and a weight.

18. A computer system, comprising:
a memory comprising,
computer readable instructions, and
a concentration measurement of a first substance and a concentration measurement of a second substance, wherein the first substance and the second substance are exhaled by a user during a time period; and
a processor configured to execute the computer readable instructions, the computer readable instructions comprising:
determine a time of interest that occurs during the time period when a change in a proportion of a first metabolic fuel type being metabolized occurs by the user based on the concentration measurement of the first substance and the concentration measurement of the second substance, and
indicate to the user the time of interest when the change in the proportion of the first metabolic fuel type being metabolized occurs.

19. The computer system of claim 18, further comprising:
receive a measurement of at least one user parameter for the time period;
determine a first rate at which the first metabolic fuel type is metabolized based on the measurement of the at least one user parameter, the concentration measurement of the first substance, and the concentration measurement of the second substance; and
indicate to the user the first rate at which the first metabolic fuel type is being metabolized.

20. The computer system of claim 18, further comprising:
receive a measurement of at least one user parameter for the time period;
receive a targeted time during the time period when the first metabolic fuel type begins being primarily metabolized;
correlate the measurement of the at least one user parameter with the time of interest;
determine whether the targeted time is substantially similar to the time of interest; and
when the time of interest is not substantially similar to the targeted time, provide a recommendation to the user on how to adjust the at least one user parameter so that the time of interest is substantially similar to the targeted time.
